(19) United States
(12) Patent Application Publication
(10) Pub. No.: US 2002/0110825 A1
(43) Pub. Date: Aug. 15, 2002

(54) Magnetic plate for biological separations

(76) Inventors: Douglas A. Spicer, Seattle, WA (US); Matt Pourfarzaneh, Alameda, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 09/992,534

(22) Filed: Nov. 16, 2001

Related U.S. Application Data
(60) Provisional application No. 60/249,568, filed on Nov. 16, 2000.

(51) Int. Cl. C12Q 1/68

(52) U.S. Cl. 435/6; 436/526; 436/531; 436/94; 436/177; 422/101; 422/102; 422/82.05

(57) ABSTRACT

This invention provides devices that are useful for applying a magnetic field to a microtiter plate. The devices find use, for example, in the removal of magnetic particles from an aqueous suspension.

### CROSS REFERENCE TO RELATED APPLICATION

This application claims the benefit of U.S. Provisional Application No. 60/249,568, filed Nov. 16, 2000, which application is incorporated herein by reference for all purposes.

### BACKGROUND OF THE INVENTION

1. Field of the Invention

This invention pertains to the field of separation of magnetic particles from a liquid phase using magnets.

2. Background

Both the rapid increase of new drug targets and the availability of vast libraries of chemical compounds creates an enormous demand for new technologies which improve the screening process. The compounds produced in the combinatorial libraries being generated by modern techniques, such as rapid parallel and automated synthesis, will far outnumber those compounds being prepared by traditional, manual means, natural product extracts, or those in the historical compound files of large pharmaceutical companies. The demands of the Human Genome Project and the commercial implications of polymorphism and gene discovery have driven the development of automated methods for DNA sequencing.

Automated multiwell formats are the best-developed high-throughput screening systems. Magnetic particles have found wide use in such microtiter well systems for the purification and analysis of biological and other substances. A ligand that binds to an analyte of interest can be attached to a magnetic particle and placed in a solution that contains the analyte and other components. After the analyte binds to the ligand, one can separate the analyte from other components by placing the solution in a magnetic field, thus concentrating the magnetic particles and permitting removal of unbound components. Numerous uses of magnetic particles for biological substances are described in U.S. Pat. No. 4,695,393 and references cited therein. Magnetic particles are also useful as supports for synthesis of organic compounds, including polynucleotides and polypeptides (see, e.g., U.S. Pat. No. 4,638,032).

Automated 96-well plate-based screening systems have been the most widely used. The current trend in plate based screening systems is to reduce the volume of the reaction wells further, thereby increasing the density of the wells per plate (96-wells to 384- and 1536-wells per plate). The reduction in reaction volumes results in increased throughput, dramatically decreased bioreagent costs, and a decrease in the number of plates which need to be managed by automation. However, the use of plates with increased well density has resulted in difficulties in concentrating magnetic particles upon placing the plates in a magnetic field.

Therefore, a need exists for improved devices for applying a magnetic field to a microtiter plate to achieve concentration of magnetic particles. The present invention fulfills this and other needs.

### BRIEF SUMMARY OF THE INVENTION

The present invention provides a device for applying a magnetic field to a microtiter plate, the device comprising a substrate and a plurality of magnetic elements disposed on the substrate, wherein the plurality of magnetic elements are arranged parallel to each other such that the longitudinal axis of each magnetic element is approximately centered under a row or column of wells of a microtiter plate when said microtiter plate is positioned upon the device.

In one specific embodiment of the invention, the magnetic elements are in contact with each other. In another specific embodiment, the magnetic elements are separated from each other by a non-magnetic material.

Certain embodiments of the present invention provide a method for removing unincorporated dye-labeled molecules from a mixture that comprises the dye-labeled molecules and a polymer into which dye-labeled molecules are incorporated, the method comprising: a) contacting the mixture with a plurality of particles that comprise a paramagnetic moiety and a porous hydrophobic material entrapped within a hydrophilic matrix; b) mixing and incubating the mixture and the particles for a sufficient time for dye-labeled molecules that are not incorporated into the polymer to pass into the hydrophilic matrix and become adsorbed onto the hydrophobic material; c) placing a microtiter plate of which one or more wells contains the mixture upon a device that comprises a plurality of magnetic elements which are arranged parallel to each other such that the longitudinal axis of each magnetic element is approximately centered under a row or column of wells of the microtiter plate, thereby concentrating the particles on a surface of the microtiter plate wells; and d) removing the liquid phase from the wells, thus leaving behind the adsorbed unincorporated dye-labeled molecules.

For a further understanding of the nature and advantages of the present invention, reference should be made to the following description in conjunction with the accompanying drawings.

### BRIEF DESCRIPTION OF THE DRAWINGS

FIG. 1 shows a top view of a 384-well microtiter plate and indicates the position of each of the wells. The wells are arrayed along 4.5 mm centers. Wells can be either cylindrical or rectangular and have either flat or concave bottoms. Each well can contain a maximum volume of approximately 120 microliters.

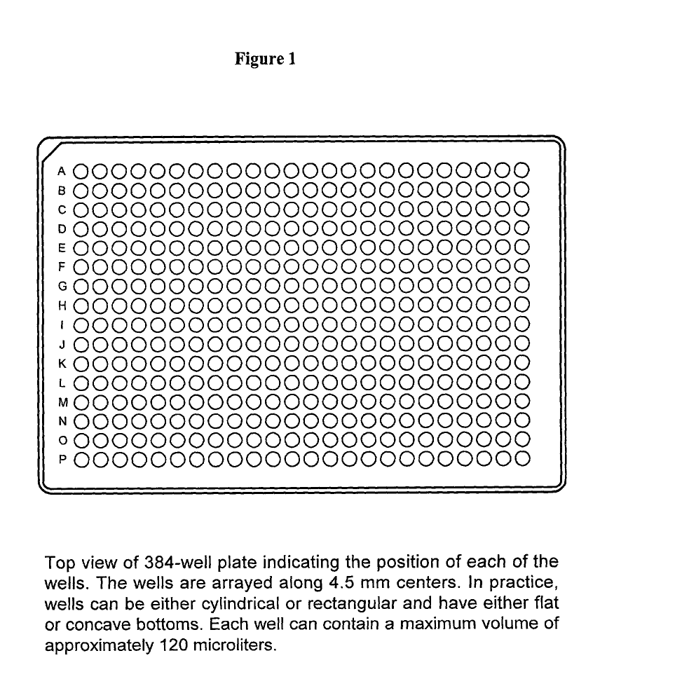

FIG. 2 shows a top view of a 384-well microtiter plate resting upon a magnetic device of the invention. The device includes 24 magnetic elements, each of which is centered about a line corresponding to each of the 24 columns of microtiter plate wells. Each magnetic element is of sufficient length to extend the length of a column.

FIG. 3 shows a top view of a 384-well microtiter plate resting upon an alternative magnetic device of the invention. The device has 16 magnetic elements, each of which is centered about a line corresponding to each of the 16 rows of microtiter plate wells. Each element is of sufficient length to extend the length of a row of microtiter plate wells.

### DETAILED DESCRIPTION OF THE INVENTION

The present invention provides magnetic devices that are useful for applying a magnetic field to wells of a microtiter plate, thereby concentrating magnetic particles that are dispensed in solution in the wells. The devices have a plurality of magnetic elements which are arranged parallel to each other such that the longitudinal axis of each magnetic element is approximately centered under a row or column of wells of a microtiter plate when the microtiter plate is positioned upon the device. The device includes a planar surface for keeping the microtiter plate horizontal.

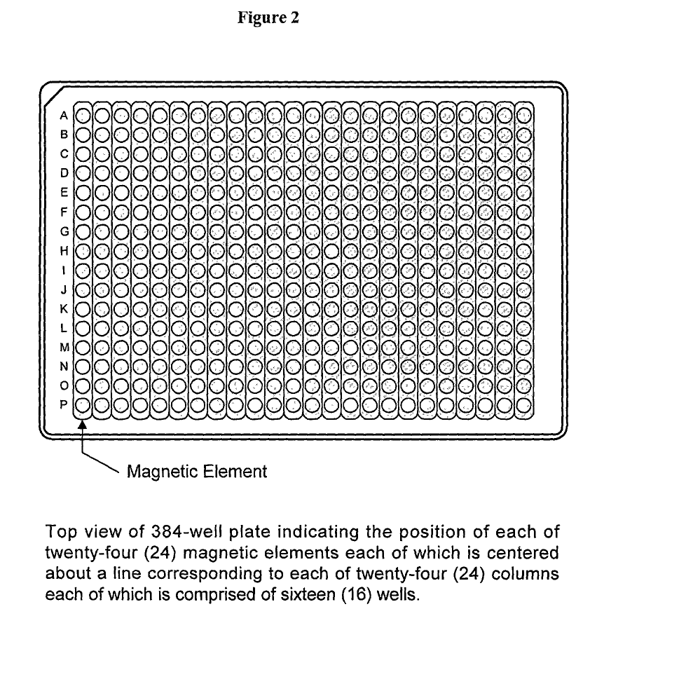

In some embodiments, the device will contain a number of magnetic elements that is equal to the number of columns of microtiter plate wells. In such embodiments, each magnetic element is preferably approximately the length of a row of microtiter plate wells. For example, as shown in FIG. 2, for use with a 384-well microtiter plate, the device includes twenty-four magnetic elements, and the longitudinal axis of each element is approximately centered under a column of wells of a 384-well microtiter plate.

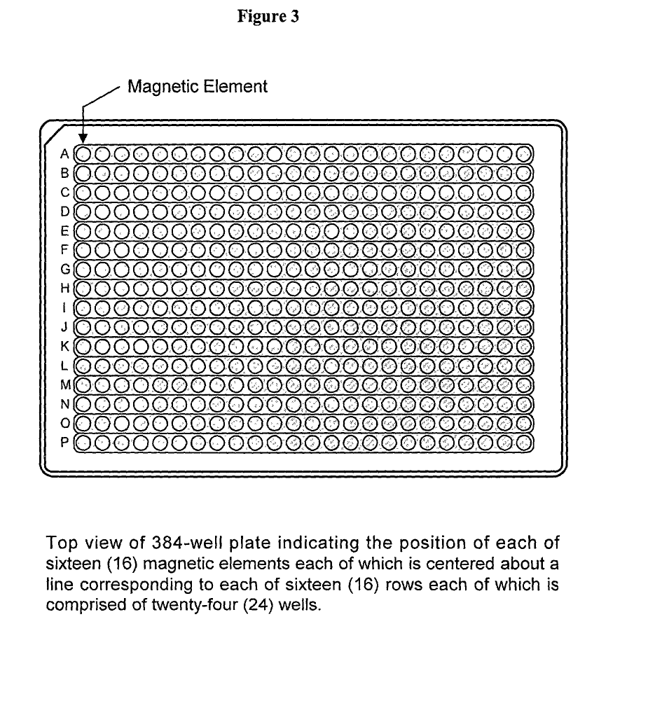

In other embodiments, the number of magnetic elements in the device is equal to the number of rows of microtiter plate wells. The length of each magnetic element in these embodiments is preferably approximately equal to the length of a column of microtiter plate wells. For example, as shown in FIG. 3, a device for use with a 384-well microtiter plate, for example, will include sixteen magnetic elements and the longitudinal axis of each element is approximately centered under a row of wells of a 384-well microtiter plate.

In either embodiment described above, the magnetic elements are placed in one of several possible arrangements, depending on the magnetic separation needs and the size and well density of the corresponding microtiter. In one embodiment, the magnetic elements are arranged such that they are in contact with one another, thereby creating a substantially spatially uniform magnetic field. In an alternate embodiment, the magnetic elements are separated from one another by a non-magnetic material, thereby providing more localized magnetic fields. In a preferred embodiment, the plurality of magnetic elements are arranged on or embedded in a substrate or solid support. Essentially, any conceivable substrate or solid support can be employed in the invention. The substrate can be organic, inorganic, biological, nonbiological, or a combination of any of these. The substrate can have any convenient shape, such a disc, square, rectangle, circle, etc. but preferably has the same shape and size of a microtiter plate (e.g., a 96- or 384-well microtiter plate). The substrate is preferably flat, but can take on a variety of alternative surface configurations. For example, the substrate can contain raised or depressed regions on which the plurality of magnetic elements are arranged. The substrate can be any of a wide variety of materials including, for example, polymers, plastics, pyrex, quartz, resins, silicon, silica or silica-based materials, carbon, metals, inorganic glasses, etc. Other substrate materials will be readily apparent to those of skill in the art upon review of this disclosure. In one (non-contacting) embodiment, the magnetic elements are placed on a nonmagnetic substrate, wherein the non-magnetic materials separating the magnetic element can include air gaps. Alternately, the magnetic materials are embedded in a substrate, and thus are separated from one another, by the material of the substrate.

Furthermore, the magnetic materials include both permanent and electromagnets. The use of electromagnets facilitates an easier activation of individual or select groups of magnetic elements. Since, the activation of electromagnets is known in the art, a more detailed description is not provided herein.

The invention also provides devices such as described herein, which also include a microtiter plate positioned upon the magnetic elements. One or more wells of the microtiter plate can contain a suspension of magnetic particles. The suspension can also include, for example, immunoassay reagents, a primer extension reaction mixture, a polymer synthesis reaction mixture, and the like. In some embodiments, the suspension comprises dye-labeled molecules and a polymer into which dye-labeled molecules are incorporated, and particles that comprise a paramagnetic moiety and a porous hydrophobic material entrapped within a hydrophilic matrix.

Also provided by the invention are methods for removing unincorporated dye-labeled molecules from a mixture that comprises the dye-labeled molecules and a polymer into which dye-labeled molecules are incorporated. These methods involve:

a) contacting the mixture with a plurality of particles that comprise a paramagnetic moiety and a porous hydrophobic material entrapped within a hydrophilic matrix;

b) mixing and incubating the mixture and the particles for a sufficient time for dye-labeled molecules that are not incorporated into the polymer to pass into the hydrophilic matrix and become adsorbed onto the hydrophobic material;

c) placing a microtiter plate of which one or more wells contains the mixture upon a device that comprises a plurality of magnetic elements which are arranged parallel to each other such that the longitudinal axis of each magnetic element is approximately centered under a row or column of wells of the microtiter plate, thereby concentrating the particles on a surface of the microtiter plate wells; and

d) removing the liquid phase from the wells, thus leaving behind the adsorbed unincorporated dye-labeled molecules.

Magnets

Suitable magnets include, for example, neodynium magnets that exhibit a magnetic field strength greater than approximately 12 KGs (Kgauss) per magnetic element. Magnets that have rare earth magnetic elements are also suitable, as are other magnets, such as electromagnets, having an appropriate magnetic field strength. An appropriate magnetic field strength is one that is of sufficient strength to be able to attract magnetically charged particles to achieve a desired separation, as described herein.

Magnetic Particles

The devices are useful for separating from a solution particles that include a magnetizable constituent. A variety of different magnetizable constituents are suitable for use in the particles. These include, for example, ferric oxide, nickel oxide, barium ferrite, and ferrous oxide.

In some embodiments, the devices of the invention are useful for removing unincorporated dye-labeled molecules from a solution that includes the dye-labeled molecules and a polymer into which dye-labeled molecules are incorporated (e.g., a primer extension reaction, such as a DNA sequencing reaction, that includes a dye-labeled primer or dideoxynucleotide). The solution is contacted with a plurality of particles that comprise a paramagnetic moiety and a porous hydrophobic material entrapped within a hydrophilic matrix; mixing and incubating the mixture and the particles for a sufficient time for dye-labeled molecules that are not incorporated into the polymer to pass into the hydrophilic matrix and become adsorbed onto the hydrophobic material. A microtiter plate that includes one or more wells that contain the mixture is placed upon the magnetic devices of the invention, thereby concentrating the particles on a surface of the microtiter plate wells. The liquid phase is then removed from the wells, thus leaving behind the adsorbed unincorporated dye-labeled molecules. Such methods are described in detail in co-pending, commonly assigned U.S. patent application Ser. No. 09/680,889, filed Oct. 6, 2000, the teachings of which are incorporated herein by reference.

Suitable magnetic particles for this application can be prepared by, for example, mixing equivalent amounts of iron oxide and hydrophobic particles (e.g., charcoal) with acrylamide/bis-acrylamide. These components are vigorously mixed to form a “cake,” which is then passed through a coffee grinder or equivalent. The ground material is then passed through a ball mill to obtain particles that are preferably about 5-20 μm in diameter.

MagaCharc™ AA particles (Cortex Biochem, San Leandro Calif.) are an example of a commercially available particle that is suitable for use in the methods of the invention. MagaCharc™ particles are prepared by cross-linking of a 1:1 mixture (w/w) of charcoal (Norit SX-Ultra) and iron oxide (Fe₃O₄) within a polymeric matrix that is prepared from 80% (w/w) polyacrylamide cross-linked with 5% N,N-methylene-bis-acrylamide and containing 15% (w/w) acrylic acid. The presence of the iron oxide distributed throughout the particle, preferably uniformly, renders the particle magnetizable and thereby facilitates the removal of supernatants from the particles by placing the particles in proximity to a magnet.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

What is claimed is:

1. A device for applying a magnetic field to a microtiter plate, said device comprising:
a substrate; and
a plurality of magnetic elements disposed on said substrate, wherein said plurality of magnetic elements are arranged parallel to each other such that the longitudinal axis of each magnetic element is approximately centered under a row or column of wells of a microtiter plate when said microtiter plate is positioned upon the device.

2. The device of claim 1, wherein said substrate is comprised of a material selected from the group consisting of polymers, plastics, pyrex, quartz, resins, silicon, silica, silica-based materials, carbon, metals, inorganic glass and combinations thereof.

3. The device of claim 1, wherein said substrate is comprised of a material selected from the group consisting of organic, inorganic, biological, nonbiological materials and combinations thereof.

4. The device of claim 1, wherein said substrate is substantially disc-shaped, square-shaped, rectangle-shaped or combinations thereof.

5. The device of claim 1, wherein said substrate has substantially the same shape and size as said microtiter plate.

6. The device of claim 1, wherein the device comprises one magnetic element for each column of wells of the microtiter plate.

7. The device of claim 1, wherein the device comprises twenty-four magnetic elements and the longitudinal axis of each element is approximately centered under a column of wells of a 384-well microtiter plate.

8. The device of claim 6, wherein each magnetic element is approximately the same length of a column of wells of the microtiter plate.

9. The device of claim 1, wherein the device comprises one magnetic element for each row of wells of the microtiter plate.

10. The device of claim 9, wherein the device comprises sixteen magnetic elements and the longitudinal axis of each element is approximately centered under a row of wells of a 384-well microtiter plate.

11. The device of claim 9, wherein each magnetic element is approximately the same length of a row of wells of the microtiter plate.

12. The device of claim 1, wherein adjacent magnetic elements are in contact with each other.

13. The device of claim 1, wherein adjacent magnetic elements are separated from on another by a non-magnetic material.

14. The device of claim 1, wherein each magnetic element is approximately as wide as the diameter of a well of the microtiter plate.

15. The device of claim 1, wherein the device does not include a mechanism for horizontal circular translation of the microtiter plate.

16. The device of claim 1, wherein the device further comprises a microtiter plate positioned upon the magnetic elements.

17. The device of claim 16, wherein one or more wells of the microtiter plate contains a suspension of magnetic particles.

18. The device of claim 17, wherein the suspension comprises immunoassay reagents.

19. The device of claim 17, wherein the suspension comprises a primer extension reaction.

20. The device of claim 19, wherein the primer extension reaction is a DNA sequencing reaction.

21. The device of claim 19, wherein the suspension comprises dye-labeled molecules and a polymer into which dye-labeled molecules are incorporated, and particles that comprise a paramagnetic moiety and a porous hydrophobic material entrapped within a hydrophilic matrix.

22. A method for removing unincorporated dye-labeled molecules from a mixture that comprises the dye-labeled molecules and a polymer into which dye-labeled molecules are incorporated, the method comprising:
a) contacting the mixture with a plurality of particles that comprise a paramagnetic moiety and a porous hydrophobic material entrapped within a hydrophilic matrix;
b) mixing and incubating the mixture and the particles for a sufficient time for dye-labeled molecules that are not incorporated into the polymer to pass into the hydrophilic matrix and become adsorbed onto the hydrophobic material;
c) placing a microtiter plate of which one or more wells contains the mixture upon a device that comprises a plurality of magnetic elements which are arranged parallel to each other such that the longitudinal axis of each magnetic element is approximately centered under a row or column of wells of the microtiter plate, thereby concentrating the particles on a surface of the microtiter plate wells; and
d) removing the liquid phase from the wells, thus leaving behind the adsorbed unincorporated dye-labeled molecules.

23. The method of claim 22, wherein the mixture comprises a primer extension reaction.

24. The method of claim 23, wherein the primer extension reaction is a DNA sequencing reaction.

25. The method of claim 24, wherein the polymers are polynucleotide molecules and the dye-labeled molecules are dye-labeled dideoxynucleotides.
